(19) United States
(12) Patent Application Publication
(10) Pub. No.: US 2025/0277330 A1
(43) Pub. Date: Sep. 4, 2025

(54) A Lofty Nonwoven Fabric

(71) Applicant: Kimberly-Clark Worldwide, Inc., Neenah, WI (US)

(72) Inventors: David A. Palzewicz, Canton, GA (US); Boshen Fu, San Ramon, CA (US); Roger B. Quincy, III, Roswell, GA (US); Laura E. Keck, Alpharetta, GA (US)

(21) Appl. No.: 18/860,728

(22) PCT Filed: May 23, 2022
(86) PCT No.: PCT/US2022/030498

§ 371 Date: Oct. 28, 2024

(51) Int. Cl. D04H 3/007; D04H 3/03; D04H 3/16

(52) U.S. Cl. CPC D04H 3/007; D04H 3/03; D04H 3/16; D10B 2509/026

(57) ABSTRACT

A nonwoven fabric includes a first plurality of randomly oriented discrete fibers and a second plurality of randomly oriented discrete fibers collectively forming a nonwoven fibrous web. The first plurality of randomly oriented discrete fibers is present within the nonwoven fibrous web at no less than seventy percent by weight of the nonwoven fibrous web, and the second plurality of randomly oriented discrete fibers is present within the nonwoven fibrous web at no less than two percent by weight of the nonwoven fibrous web and no greater than twenty percent by weight of the nonwoven fibrous web. A portion of the nonwoven fibrous web is embossed.

### BACKGROUND

Nonwoven fabrics are frequently embossed to add surface features, such as patterns or textures. In recent years, the speed at which nonwoven fabrics can be embossed has increased significantly. However, embossing the nonwoven fabric to have a high loft or thickness at the increased speeds has been challenging. Moreover, when embossed at the high speeds, the loft of the nonwoven fabric can be limited to an undesirably low value.

A nonwoven material with features that facilitate formation of lofty embosses on the nonwoven material would be useful. Moreover, a nonwoven material with features that facilitate formation of lofty embosses at high speeds would be useful.

### SUMMARY

In general, the present disclosure is directed to a nonwoven fabric with features that facilitate formation of lofty embosses. The nonwoven fabric may include a nonwoven fibrous web collectively formed from a first plurality of randomly oriented discrete fibers and a second plurality of randomly oriented discrete fibers. The second plurality of randomly oriented discrete fibers may be smaller than the first plurality of randomly oriented discrete fibers. For example, the second plurality of randomly oriented discrete fibers may be meltblown fibers, and the first plurality of randomly oriented discrete fibers may be spunbound fibers. Thus, e.g., the second plurality of randomly oriented discrete fibers may be in a microfiber size range. The second plurality of randomly oriented discrete fibers may be present in the nonwoven fibrous web at no less than two percent (2%) by weight and no greater than twenty percent (20%) by weight of the nonwoven fibrous web. The second plurality of randomly oriented discrete fibers may assist with holding the nonwoven fibrous web in a desired loft, such as a 3D shape, when the nonwoven fibrous web is embossed at high speeds, e.g., between two hundred meters per second (200 m/s) and one thousand meters per second (1000 m/s). Thus, the nonwoven fibrous web may be embossed or thermally bonded while advantageously maintaining the desired loft. The “lofty” embossed fabric may be incorporated within an absorbent articles, such as a pad, diaper, disposable undergarment, etc.

In one example embodiment, a nonwoven fabric includes a first plurality of randomly oriented discrete fibers and a second plurality of randomly oriented discrete fibers collectively forming a nonwoven fibrous web. A ratio of a mean diameter of the first plurality of randomly oriented discrete fibers to a mean diameter of the second plurality of randomly oriented discrete fibers is between 50:1 and 3:1. The first plurality of randomly oriented discrete fibers is present within the nonwoven fibrous web at no less than seventy percent by weight of the nonwoven fibrous web, and the second plurality of randomly oriented discrete fibers is present within the nonwoven fibrous web at no less than two percent by weight of the nonwoven fibrous web and no greater than twenty percent by weight of the nonwoven fibrous web. A portion of the nonwoven fibrous web is embossed.

In a first example aspect, the first plurality of randomly oriented discrete fibers may be polypropylene fibers, and the second plurality of randomly oriented discrete fibers may be polypropylene fibers.

In a second example aspect, the first plurality of randomly oriented discrete fibers may be spunbound polypropylene fibers, and the second plurality of randomly oriented discrete fibers may be meltblown polypropylene fibers.

In a third example aspect, the mean diameter of the first plurality of randomly oriented discrete fibers may be no less than ten microns and no greater than twenty-two microns, and the mean diameter of the second plurality of randomly oriented discrete fibers may be no less than a half micron and no greater than three microns.

In a fourth example aspect, the mean diameter of the first plurality of randomly oriented discrete fibers is no less than twelve microns and no greater than eighteen microns, and the mean diameter of the second plurality of randomly oriented discrete fibers is no less than seven-tenths of a micron and no greater than two microns.

In a fifth example aspect, a mean thickness of the nonwoven fibrous web at an unembossed portion of the nonwoven fibrous web may be no less than three times greater than a mean thickness of the nonwoven fibrous web at the embossed portion of the nonwoven fibrous web.

In a sixth example aspect, the mean thickness of the nonwoven fibrous web at the unembossed portion of the nonwoven fibrous web may be no less than five times greater than the mean thickness of the nonwoven fibrous web at the embossed portion of the nonwoven fibrous web.

In a seventh example aspect, the mean thickness of the nonwoven fibrous web at the unembossed portion of the nonwoven fibrous web may be no less than one hundred microns and no greater than about three hundred microns, and the mean thickness of the nonwoven fibrous web at the embossed portion of the nonwoven fibrous web may be no less than five hundred microns and no greater than three thousand microns.

In an eighth example aspect, the nonwoven fibrous web may define a plurality of apertures that extend through a thickness of the nonwoven fibrous web.

In a nineth example aspect, a product may include the embossed nonwoven fabric. The product may be one of a diaper, a disposable undergarment, an incontinence pad, and a feminine hygiene pad.

Each of the example aspects recited above may be combined with one or more of the other example aspects recited above in certain embodiments. For instance, all of the nine example aspects recited above may be combined with one another in some embodiments. As another example, any combination of two, three, four, five, or more of the nine example aspects recited above may be combined in other embodiments. Thus, the example aspects recited above may be utilized in combination with one another in some example embodiments. Alternatively, the example aspects recited above may be individually implemented in other example embodiments. Accordingly, it will be understood that various example embodiments may be realized utilizing the example aspects recited above.

In another example embodiment, a nonwoven fabric includes a plurality of randomly oriented discrete spunbound polypropylene fibers and a plurality of randomly oriented discrete meltblown polypropylene fibers collectively forming a nonwoven fibrous web. A ratio of a mean diameter of the plurality of randomly oriented discrete spunbound polypropylene fibers to a mean diameter of the plurality of randomly oriented discrete meltblown polypropylene fibers is between 60:1 and 2:1. The plurality of randomly oriented discrete spunbound polypropylene fibers is present within the nonwoven fibrous web at no less than seventy percent by weight of the nonwoven fibrous web, and the plurality of randomly oriented discrete meltblown polypropylene fibers is present within the nonwoven fibrous web at no less than two percent by weight of the nonwoven fibrous web and no greater than twenty percent by weight of the nonwoven fibrous web. A portion of the nonwoven fibrous web is embossed such that a mean thickness of the nonwoven fibrous web at an unembossed portion of the nonwoven fibrous web is no less than three times greater than a mean thickness of the nonwoven fibrous web at the embossed portion of the nonwoven fibrous web.

In a tenth example aspect, the mean diameter of the plurality of randomly oriented discrete spunbound polypropylene fibers may be no less than ten microns and no greater than twenty-two microns, and the mean diameter of the plurality of randomly oriented discrete meltblown polypropylene fibers may be no less than a half micron and no greater than three microns.

In an eleventh example aspect, the mean diameter of the plurality of randomly oriented discrete spunbound polypropylene fibers may be no less than twelve microns and no greater than eighteen microns, and the mean diameter of the plurality of randomly oriented discrete meltblown polypropylene fibers may be no less than seven-tenths of a micron and no greater than two microns.

In a twelfth example aspect, the mean thickness of the nonwoven fibrous web at the unembossed portion of the nonwoven fibrous web may be no less than five times greater than the mean thickness of the nonwoven fibrous web at the embossed portion of the nonwoven fibrous web.

In a thirteenth example aspect, the mean thickness of the nonwoven fibrous web at the unembossed portion of the nonwoven fibrous web may be no less than one hundred microns and no greater than about three hundred microns, and the mean thickness of the nonwoven fibrous web at the embossed portion of the nonwoven fibrous web may be no less than five hundred microns and no greater than three thousand microns.

In a fourteenth example aspect, the nonwoven fibrous web may define a plurality of apertures that extend through a thickness of the nonwoven fibrous web.

Each of the example aspects recited above may be combined with one or more of the other example aspects recited above in certain embodiments. For instance, all of the five example aspects recited above (i.e., the tenth through fourteenth example aspects) may be combined with one another in some embodiments. As another example, any combination of two, three, or four of the five example aspects recited above may be combined in other embodiments. Thus, the example aspects recited above may be utilized in combination with one another in some example embodiments. Alternatively, the example aspects recited above may be individually implemented in other example embodiments. Accordingly, it will be understood that various example embodiments may be realized utilizing the example aspects recited above.

In an additional example embodiment, a method for forming an embossed nonwoven fabric, includes: bonding a mixture of a plurality of randomly oriented discrete spunbound polypropylene fibers and a plurality of randomly oriented discrete meltblown polypropylene fibers to form a nonwoven fibrous web, the plurality of randomly oriented discrete spunbound polypropylene fibers present within the mixture at no less than seventy percent by weight of the mixture, the plurality of randomly oriented discrete meltblown polypropylene fibers present within the mixture at no less than two percent by weight of the mixture and no greater than twenty percent by weight of the mixture; and embossing a portion of the nonwoven fibrous web such that a mean thickness of the nonwoven fibrous web at an unembossed portion of the nonwoven fibrous web is no less than three times greater than a mean thickness of the nonwoven fibrous web at the embossed portion of the nonwoven fibrous web.

In a fifteenth example aspect, a speed of the nonwoven fibrous web during the embossing may be no less than two hundred meters per second and no greater than one thousand meters per second

In a sixteenth example aspect, a ratio of a mean diameter of the plurality of randomly oriented discrete spunbound polypropylene fibers to a mean diameter of the plurality of randomly oriented discrete meltblown polypropylene fibers may be between 50:1 and 3:1.

The fifteenth and sixteenth examples aspects may be combined in certain example embodiments.

These and other features, aspects and advantages of the present disclosure will become better understood with reference to the following description and appended claims. The accompanying drawings, which are incorporated in and constitute a part of this specification, illustrate embodiments of the disclosure and, together with the description, serve to explain the principles of the disclosure.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures.

FIG. 1 is a side, elevation view of a nonwoven fabric according to an example embodiment of the present disclosure.

FIG. 2 is a top, plan view of the example nonwoven fabric of FIG. 1.

FIG. 3 is a schematic view of an embosser for a nonwoven fabric according to an example embodiment of the present disclosure.

FIG. 4 is a partial section view the example embosser of FIG. 3.

FIG. 5 illustrates a method for forming a nonwoven fabric according to an example embodiment of the present disclosure.

FIG. 6 is scan of a nonwoven fabric according to an example embodiment of the present disclosure.

FIG. 7 is a plot of a thickness of the nonwoven fabric of FIG. 6.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

As used herein, the terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a ten percent (10%) margin.

As used herein, the term “fiber” refers to any type of artificial fiber, filament, or fibril, whether continuous or discontinuous, produced through a spinning process, a meltblowing process, a melt fibrillation or film fibrillation process, or an electrospinning production process, or any other suitable process. As used herein, the term “nonwoven” means a porous, fibrous material made from continuous (long) filaments (fibers) and/or discontinuous (short) filaments (fibers) by processes such as, for example, spunbonding, meltblowing, carding, and the like. “Nonwoven” does not include a film, woven cloth, or knitted cloth.

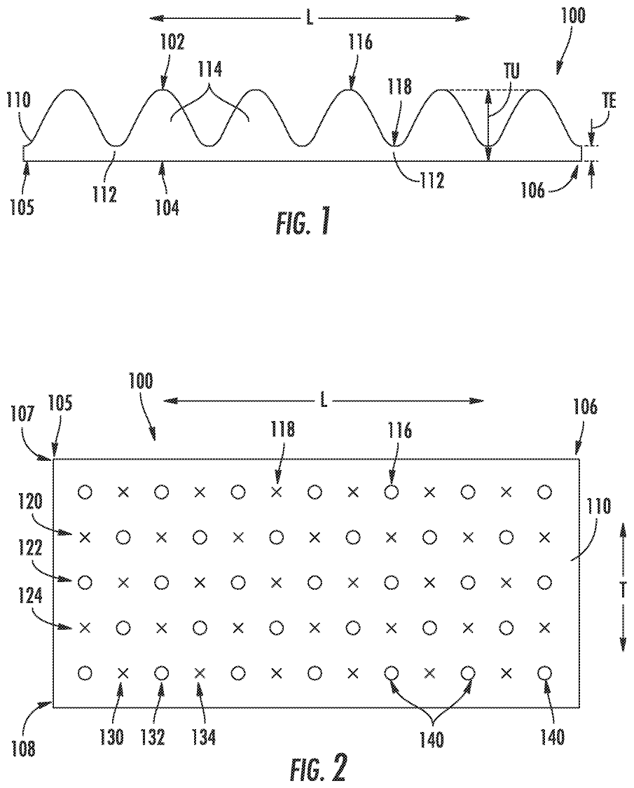

With reference to FIGS. 1 and 2, a nonwoven fabric 100 according to an example embodiment of the present disclosure is shown. Nonwoven fabric 100 may include a nonwoven fibrous web 110 formed from a first plurality of randomly oriented discrete fibers and a second plurality of randomly oriented discrete fibers that are bonded together. The first and second pluralities of randomly oriented discrete fibers may have different sizes. Moreover, the first plurality of randomly oriented discrete fibers may larger than the second plurality of randomly oriented discrete fibers. For example, a mean diameter of the first plurality of randomly oriented discrete fibers may be greater than a mean diameter of the second plurality of randomly oriented discrete fibers. As described in greater detail below, the sizing difference between the first and second pluralities of randomly oriented discrete fibers may advantageously assist the nonwoven fibrous web 110 with maintaining a desirable loft during bonding and/or embossing of nonwoven fibrous web 110.

As shown in FIGS. 1 and 2, nonwoven fibrous web 110 may be embossed to provide a desired pattern and/or texture on nonwoven fibrous web 110. Moreover, nonwoven fibrous web 110 may define a first face 102 and a second face 104, e.g., positioned opposite each other on nonwoven fibrous web 110. Nonwoven fibrous web 110 may be embossed to form embossed portions 112 and lofted portions 116 on nonwoven fibrous web 110. For example, the embossed portions 112 may correspond to troughs or valleys 118 (represented with the “X”s in FIG. 2) at first face 102 of nonwoven fibrous web 110, and lofted portions 116 may correspond to projections or peaks 116 (represented with the “O”s in FIG. 2) at first face 102 of nonwoven fibrous web 110. As shown in FIG. 2, embossed portions 112 and lofted portions 116 may be distributed in a plurality of rows 120, 122, 124 and/or a plurality of columns 130, 132, 134 at first face 102 of nonwoven fibrous web 110. Nonwoven fibrous web 110 may extend between a first end portion 105 and a second end portion 106, e.g., along a longitudinal direction L. Nonwoven fibrous web 110 may also extend between a first side portion 107 and a second side portion 108, e.g., along a transverse direction T, which is perpendicular to the lateral direction L. Rows 120, 122, 124 may be spaced apart along transverse direction T between first and second side portions 107, 108 of nonwoven fibrous web 110, and columns 130, 132, 134 may be spaced apart along lateral direction L between first and second end portions 105, 106 of nonwoven fibrous web 110. It will be understood that such example pattern is provided by way of example only and is not intended to limit the present disclose to any particular arrangement of embossed portions 112 and lofted portions 116. Rather, nonwoven fibrous web 110 may be embossed to provide any suitable pattern and/or texture of embossed portions 112 and lofted portions 116 on nonwoven fibrous web 110.

A loft or thickness of nonwoven fibrous web 110 may be different at embossed portions 112 and lofted portions 116. In particular, the thickness of nonwoven fibrous web 110 at lofted portions 116 may be greater than the thickness of nonwoven fibrous web 110 at embossed portions 112. Moreover, a mean thickness TU of nonwoven fibrous web 110 at lofted portions 116 of nonwoven fibrous web 110 may be no less than three times (3×) greater than a mean thickness TE of nonwoven fibrous web 110 at embossed portions 112 of nonwoven fibrous web 110. As another example, the mean thickness TU of nonwoven fibrous web 110 at lofted portions 116 may be no less than four times (4×) greater, five times (5×) greater, six times (6×) greater, ten times (10×) greater, twenty times (20×) greater, thirty times (6×) greater, or more than the mean thickness TE of nonwoven fibrous web 110 at embossed portions 112. In certain example embodiments, the mean thickness TU of nonwoven fibrous web 110 at lofted portions 116 may be no less than one hundred microns (100 μm) and no greater than about three hundred microns (300 μm), and the mean thickness TE of nonwoven fibrous web 110 at embossed portions 112 may be no less than five hundred microns (500 μm) and no greater than three thousand microns (3000 μm). As may be seen from the above, nonwoven fibrous web 110 may advantageously have a significant difference in thickness between the embossed portions 112 and lofted portions 116 of nonwoven fibrous web 110. Thus, e.g., nonwoven fabric 100 may be “lofty” and/or include a “3D” textured surface.

As shown, the mean thickness TU of nonwoven fibrous web 110 at lofted portions 116 of nonwoven fibrous web 110 may correspond to the thickness of nonwoven fibrous web 110 between first face 102 and second face 104 at lofted portions 116. Moreover, the relevant thickness may be the maximum thickness of nonwoven fibrous web 110 between first face 102 and second face 104 at each lofted portion 116. Conversely, the mean thickness TE of nonwoven fibrous web 110 at embossed portions 112 of nonwoven fibrous web 110 may correspond to the thickness of nonwoven fibrous web 110 between first face 102 and second face 104 at embossed portions 112. Moreover, the relevant thickness may be the minimum thickness of nonwoven fibrous web 110 between first face 102 and second face 104 at each embossed portion 112.

The first and second pluralities of randomly oriented discrete fibers may be formed from a suitable plastic material. For example, the first plurality of randomly oriented discrete fibers may be extruded polypropylene fibers, extruded polyethylene fibers, bonded carded web (BCW), etc. In certain example embodiments, the first plurality of randomly oriented discrete fibers may be spunbond polypropylene fibers. The second plurality of randomly oriented discrete fibers may also be extruded polypropylene fibers, extruded polyethylene fibers, bonded carded web (BCW), etc. In certain example embodiments, the second plurality of randomly oriented discrete fibers may be meltblown polypropylene fibers.

As noted above, the first plurality of randomly oriented discrete fibers may be relatively large as compared to the second plurality of randomly oriented discrete fibers. For instance, a ratio of a mean diameter of the first plurality of randomly oriented discrete fibers to a mean diameter of the second plurality of randomly oriented discrete fibers may be between 50:1 and 3:1, such as between 26:1 and 6:1. In certain example embodiments, the mean diameter of the first plurality of randomly oriented discrete fibers may be greater than the mean diameter of the second plurality of randomly oriented discrete fibers. As a particular example, the mean diameter of the first plurality of randomly oriented discrete fibers may be no less than ten microns (10 μm) and no greater than twenty-two microns (22 μm). More particularly, the mean diameter of the first plurality of randomly oriented discrete fibers may be no less than twelve microns (12 μm) and no greater than eighteen microns (18 μm). In certain example embodiments, the mean diameter of the second plurality of randomly oriented discrete fibers may be no less than a half micron (0.5 μm) and no greater than three microns (3 μm). More particularly, the mean diameter of the second plurality of randomly oriented discrete fibers may be no less than seven-tenths (0.7 μm) of a micron and no greater than two microns (2 μm). Such differential sizing of the first and second pluralities of randomly oriented discrete fibers may advantageously assist with allowing formation of nonwoven fibrous web 110 with the significant difference in thickness between the embossed portions 112 and lofted portions 114 of nonwoven fibrous web 110.

The first and second pluralities of randomly oriented discrete fibers may be mixed together and bonded to form nonwoven fibrous web 110. The relative amounts of the first and second pluralities of randomly oriented discrete fibers within the nonwoven fibrous web 110 may be selected to assist with forming nonwoven fibrous web 110 with the significant difference in thickness between the embossed portions 112 and lofted portions 114 of nonwoven fibrous web 110. The first plurality of randomly oriented discrete fibers may be present within nonwoven fibrous web 110 at no less than seventy percent (70%), no less than eighty percent (80%), no less than ninety percent (90%), no less than ninety-five percent (95%), or more by weight of the nonwoven fibrous web 110. In certain example embodiments, the first plurality of randomly oriented discrete fibers may be present within nonwoven fibrous web 110 at no less than seventy percent (70%) and no greater than ninety-seven percent (97%) by weight of the nonwoven fibrous web 110. The second plurality of randomly oriented discrete fibers may be present within nonwoven fibrous web 110 at no less than two percent (2%), no less than three percent (3%), no less than five percent (5%), no less than eight percent (8%), no less than ten percent (10%), or no less than twelve percent (12%) by weight of the nonwoven fibrous web 110. The second plurality of randomly oriented discrete fibers may be present within nonwoven fibrous web 110 at no greater than five percent (5%), no greater than eight percent (8%), no greater than ten percent (10%), no greater than twelve percent (12%), no greater than fifteen percent (15%), no greater than eighteen percent (18%), or no greater than twenty percent (20%) by weight of the nonwoven fibrous web 110. In certain example embodiments, the second plurality of randomly oriented discrete fibers may be present within nonwoven fibrous web 110 at no less than two percent (2%) and no greater than twenty percent (20%) by weight of the nonwoven fibrous web 110, such as no less than three percent (3%) and no greater than fifteen percent (15%) by weight of the nonwoven fibrous web 110. Such concentration of the second plurality of randomly oriented discrete fibers within the nonwoven fibrous web 110 may advantageously assist with allowing formation of nonwoven fibrous web 110 with the significant difference in thickness between the embossed portions 112 and lofted portions 114 of nonwoven fibrous web 110.

As shown in FIG. 3, nonwoven fibrous web 110 may also define a plurality of apertures 140. Apertures 140 may extend through nonwoven fibrous web 110, e.g., between first and second faces 102, 104 of nonwoven fibrous web 110. Apertures may allow liquids to pass through nonwoven fibrous web 110, e.g., from first face 102 to second face 104 or vice versa.

Nonwoven fibrous web 110 may be used to at least partially form a product, such as a diaper, a disposable undergarment, an incontinence pad, a feminine hygiene pad, etc. For example, nonwoven fibrous web 110 may be in a diaper as an inner lining, e.g., that contacts the skin of a wearer. The significant difference in thickness between the embossed portions 112 and lofted portions 114 of nonwoven fibrous web 110 may increase a comfort of the wearer relative to known linings. For instance, distal ends of the lofted portions 114 may contact the wearer, and air may fill the gaps formed at the embossed portions 112. In addition, liquids and other matter may flow away from the wearer through the gaps formed at the embossed portions 112 and/or apertures 140. The space provided by the gaps formed at the embossed portions 112 may advantageously limit discomfort by allowing liquids and other matter to move away from the wearer and/or the air within the gaps formed at the embossed portions 112 may facilitate drying of the skin of the wearer. The “lofty” characteristics of the nonwoven fibrous web 110 may thus advantageously increase a comfort of the wearer.

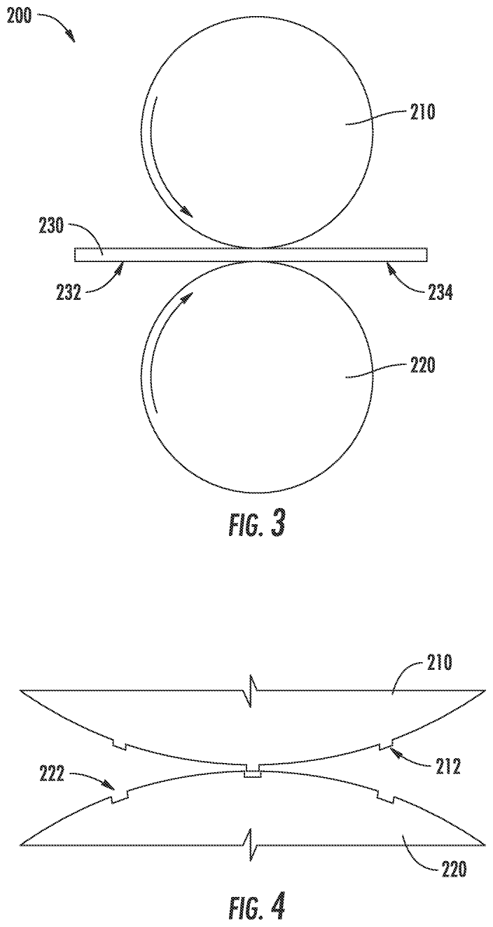

Turning now to FIGS. 3 and 4, a schematic view of an embosser 200 for a nonwoven fabric according to an example embodiment of the present disclosure. Embosser 200 may be used to emboss nonwoven fibrous web 110, e.g., in order to form embossed portions 112 and lofted portions 114 on nonwoven fibrous web 110. However, it will be understood that embosser 200 may be used to emboss other suitable nonwoven fabrics in alternative example embodiments.

As shown in FIGS. 3 and 4, embosser 200 includes a first roller 210 and a second roller 220. A nonwoven fibrous web 230 may pass between first and second rollers 210, 220 during operation of embosser 200 in order to emboss nonwoven fibrous web 230. The nonwoven fibrous web 230 is not embossed upstream of first and second rollers 210, 220 (indicated with arrow 232) and is embossed downstream of first and second rollers 210, 220 (indicated with arrow 234), First and second rollers 210, 220 may rotate to move nonwoven fibrous web 230 through embosser 200.

As shown in FIG. 4, first roller 210 may include a plurality of projections 212, and second roller 220 may define a plurality of cavities 222. Each projection 212 of first roller 210 may be received within a respective cavity 222 of second roller 220 during rotation of first and second rollers 210, 220. The shape and placement of projections 212 and/or cavities 222 may correspond to the shape and placement of embossments on nonwoven fibrous web 230, such as embossed portions 112 and lofted portions 114 on nonwoven fibrous web 110. First roller 210 and/or second roller 220 may be heated during operation of embosser 200 to assist with forming embossments on nonwoven fibrous web 230.

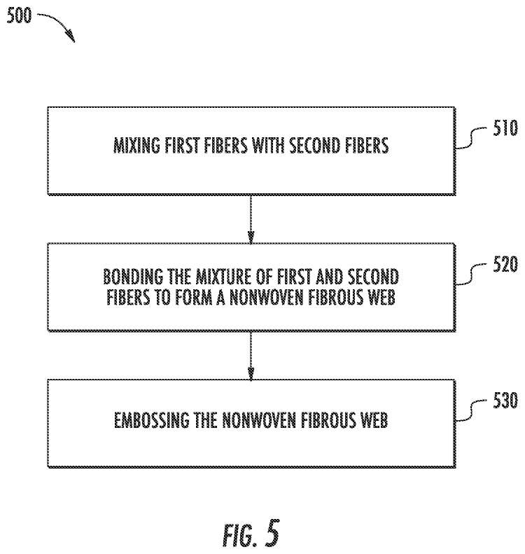

FIG. 5 illustrates a method 500 for forming a nonwoven fabric according to an example embodiment of the present disclosure. As an example, method 500 may be used to form nonwoven fabric 100 with the characteristic described above for nonwoven fabric 100. Thus, method 500 is described in greater detail below in the context of nonwoven fabric 100. However, it will be understood that method 500 may be used to form other nonwoven fabrics in alternative example embodiments. As discussed in greater detail below, utilizing method 500 may advantageously provide a “lofty” nonwoven fabric.

At 510, the first plurality of randomly oriented discrete fibers may be mixed with the second plurality of randomly oriented discrete fibers. For instance, with respect to the first plurality of randomly oriented discrete fibers, pellets of plastic (e.g., polypropylene) may be extruded through a large number of orifices in a spinneret or die to form fine filaments that are rapidly drawn and collected on a belt in order to form the first plurality of randomly oriented discrete fibers. Thus, the first plurality of randomly oriented discrete fibers may be spunbond. As another example, with respect to the second plurality of randomly oriented discrete fibers, pellets of plastic (e.g., polypropylene) may be extruded through small nozzles surrounded by high-speed blowing gas to form fine filaments, which are blow onto a belt in order to form the second plurality of randomly oriented discrete fibers. Thus, the second plurality of randomly oriented discrete fibers may be meltblown. The first plurality of randomly oriented discrete fibers may be mixed with the second plurality of randomly oriented discrete fibers by “dusting” the first plurality of randomly oriented discrete fibers with the second plurality of randomly oriented discrete fibers, which are relatively smaller.

At 520, the mixture of the first and second pluralities of randomly oriented discrete fibers may be bonded to form the nonwoven fibrous web 110. For instance, the mixture of the first and second pluralities of randomly oriented discrete fibers may be conveyed through a thermal bonder to form the nonwoven fibrous web 110 from the mixture of the first and second pluralities of randomly oriented discrete fibers.

At 530, a portion of nonwoven fibrous web 110 formed at 520 may be embossed. Thus, after 530, nonwoven fibrous web 110 may include embossed portions 112 and lofted portions 114. Due to the concentration of the second plurality of randomly oriented discrete fibers within the nonwoven fibrous web 110, the mean thickness TU of nonwoven fibrous web 110 at lofted portions 114 of nonwoven fibrous web 110 may be no less than three times (3×) greater than the mean thickness TE of nonwoven fibrous web 110 at embossed portions 112 of nonwoven fibrous web 110 after 530. It will be understood that the bonding at 520 and the embossing at 530 may be combined into a single step in certain example embodiments. A speed of the nonwoven fibrous web 110 through an embosser at 530, e.g., through embosser 200, may be no less than two hundred meters per second (200 m/s) and no greater than one thousand meters per second (1000 m/s). Accordingly, method 500 may advantageously form lofty embossments on nonwoven fibrous web 110 despite a high processing speed during embossing of the nonwoven fibrous web 110.

FIG. 5 depicts steps performed in a particular order for purposes of illustration and discussion. Those of ordinary skill in the art, using the disclosures provided herein, will understand that the steps of any of the methods discussed herein may be adapted, rearranged, expanded, omitted, or modified in various ways without deviating from the scope of the present disclosure.

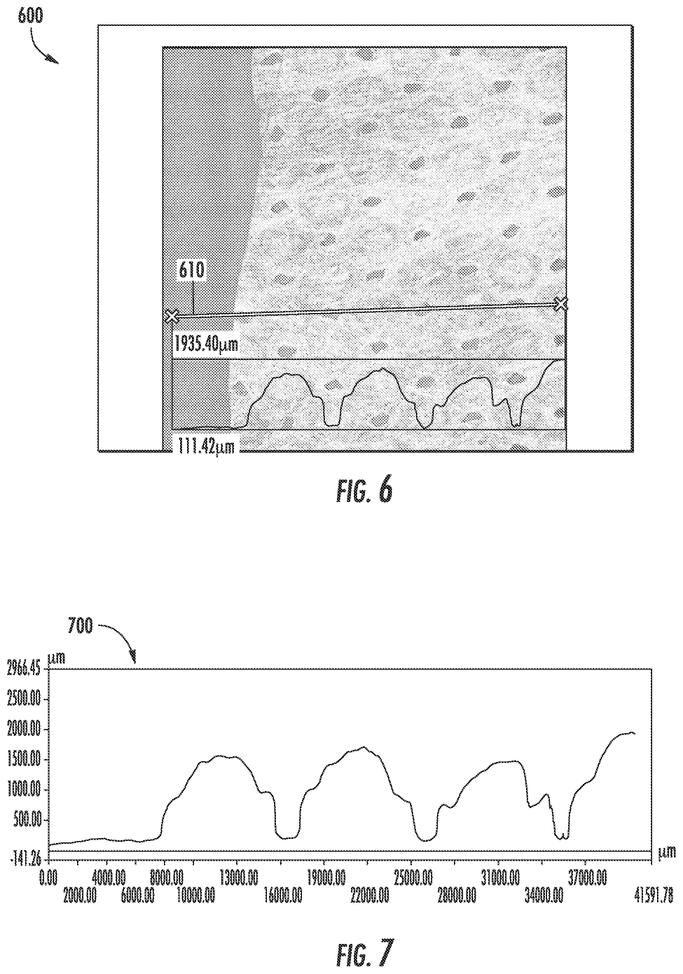

FIG. 6 is scan 600 of a nonwoven fabric according to an example embodiment of the present disclosure. Moreover, FIG. 6 shows an image from a scanning electron microscope of a nonwoven fabric according to an example embodiment of the present disclosure. The example nonwoven fabric shown in scan 600 was formed via method 500. As shown in FIG. 6, the nonwoven fabric includes various embossments, which are the darker circles within the nonwoven fabric. Measurements of the thickness of the nonwoven fabric were taken along the line 610 in FIG. 6. FIG. 7 is a plot of the thickness of the nonwoven fabric taken along the line 610. As shown, the thickness of the nonwoven fabric increases and decreases between adjacent embossments. Moreover, a maximum measured thickness of the nonwoven fabric was about two thousand microns (2000 μm), and a minimum measured thickness of the nonwoven fabric was about one hundred and eleven microns (111 μm). Thus, the example nonwoven fabric advantageously has “lofty” characteristics despite being embossed at high speeds.

Herein, the diameter of fibers in a sample of a nonwoven fibrous web may be determined by using a Scanning Electron Microscope (SEM) and image analysis software. A magnification of five hundred (500) to ten thousand (10,000) times may be chosen such that the fibers are suitably enlarged for measurement. The samples may be sputtered with gold or a palladium compound to avoid electric charging and vibrations of the fibers in the electron beam. A manual procedure for determining the fiber diameters may be used. Using a mouse and a cursor tool, the edge of a randomly selected fiber may be sought and then measured across the width of the fiber (i.e., perpendicular to fiber direction at that point) to the other edge of the fiber. A scaled and calibrated image analysis tool may provide the scaling to get actual measurement in microns (μm). Several fibers may thus be randomly selected across the sample of the nonwoven fibrous web using the SEM. At least two specimens from the nonwoven fibrous web (or nonwoven fibrous web inside a product) may be cut and tested in this manner. Altogether at least one hundred (100) such measurements may be made and then all data may be recorded for statistic analysis. The recorded data may be used to calculate the mean diameter of the fibers, standard deviation of the fiber diameter, and median of the fiber diameter. Another useful statistic is the calculation of the amount of the population of fibers that is below a certain upper limit. To determine this statistic, the software is programmed to count how many results of the fiber diameters are below an upper limit and that count (divided by total number of data and multiplied by 100%) is reported in percent as percent below the upper limit, such as percent below one micron diameter or %-submicron, for example.

Herein, the thickness of a sample nonwoven fibrous web may be determined by using a Scanning Electron Microscope (SEM) and image analysis software, e.g., as shown above in FIGS. 6 and 7. A magnification of five hundred (500) to ten thousand (10,000) times may be chosen such that the nonwoven fibrous web is suitably enlarged for measurement. The sample may be sputtered with gold or a palladium compound to avoid electric charging and vibrations of the fibers of the nonwoven fibrous web in the electron beam. A manual procedure for determining the web thickness may be used. Using a mouse and a cursor tool, a line across a randomly selected portion of the web may be sought and then the web thickness along the line may be measured, e.g., from a base on which the web is positioned to a closest surface of the web. A scaled and calibrated image analysis tool may provide the scaling to get actual measurement in microns (μm). Several sections of the web may thus be randomly selected across the sample web using the SEM. At least two specimens from the nonwoven fibrous web (or nonwoven fibrous web inside a product) may be cut and tested in this manner. Altogether at least one hundred (100) such measurements may be made and then all data may be recorded for statistic analysis. The recorded data may be used to calculate the mean thickness of the web, standard deviation of the web thickness, and median of the web thickness. Another useful statistic is the calculation of the portion of measured web thicknesses in the sample web that is below a certain upper limit. To determine this statistic, the software is programmed to count how many results of the web thicknesses are below an upper limit and that count (divided by total number of data and multiplied by 100%) is reported in percent as percent below the upper limit, such as percent below one micron diameter or %-submicron, for example.

Repeat use of reference characters in the present specification and drawings is intended to represent the same or analogous features or elements of the present invention.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

### REFERENCE CHARACTERS

100 Nonwoven fabric
102 First face
104 Second face
105 First end portion
106 Second end portion
107 First side portion
108 Second side portion
110 Nonwoven fibrous web
112 Embossed portions
114 Lofted portions
116 Peaks
118 Floors
120 First row
122 Second row
124 Third row
130 First column
132 Second column
134 Third column
140 Apertures
200 Embosser
210 First roller
212 Projections
220 Second roller
222 Cavities
500 Method
510 Step
520 Step
530 Step
600 Scan
700 Plot

### Example Embodiments

First example embodiment: A nonwoven fabric, comprising a first plurality of randomly oriented discrete fibers and a second plurality of randomly oriented discrete fibers collectively forming a nonwoven fibrous web. A ratio of a mean diameter of the first plurality of randomly oriented discrete fibers to a mean diameter of the second plurality of randomly oriented discrete fibers is between 50:1 and 3:1. The first plurality of randomly oriented discrete fibers is present within the nonwoven fibrous web at no less than seventy percent by weight of the nonwoven fibrous web. The second plurality of randomly oriented discrete fibers is present within the nonwoven fibrous web at no less than two percent by weight of the nonwoven fibrous web and no greater than twenty percent by weight of the nonwoven fibrous web. A portion of the nonwoven fibrous web is embossed.

Second example embodiment: The embossed nonwoven fabric of the first example embodiment, wherein the first plurality of randomly oriented discrete fibers are polypropylene fibers, and the second plurality of randomly oriented discrete fibers are polypropylene fibers.

Third example embodiment: The embossed nonwoven fabric of either the first example embodiment or the second example embodiment, wherein the first plurality of randomly oriented discrete fibers are spunbound polypropylene fibers, and the second plurality of randomly oriented discrete fibers are meltblown polypropylene fibers.

Fourth example embodiment: The embossed nonwoven fabric of any one of the first through third example embodiments, wherein: the mean diameter of the first plurality of randomly oriented discrete fibers is no less than ten microns and no greater than twenty-two microns; and the mean diameter of the second plurality of randomly oriented discrete fibers is no less than a half micron and no greater than three microns.

Fifth example embodiment: The embossed nonwoven fabric of any one of the first through fourth example embodiments, wherein: the mean diameter of the first plurality of randomly oriented discrete fibers is no less than twelve microns and no greater than eighteen microns; and the mean diameter of the second plurality of randomly oriented discrete fibers is no less than seven-tenths of a micron and no greater than two microns.

Sixth example embodiment: The embossed nonwoven fabric of any one of the first through fifth example embodiments, wherein a mean thickness of the nonwoven fibrous web at an unembossed portion of the nonwoven fibrous web is no less than three times greater than a mean thickness of the nonwoven fibrous web at the embossed portion of the nonwoven fibrous web.

Seventh example embodiment: The embossed nonwoven fabric of any one of the first through sixth example embodiments, wherein the mean thickness of the nonwoven fibrous web at the unembossed portion of the nonwoven fibrous web is no less than five times greater than the mean thickness of the nonwoven fibrous web at the embossed portion of the nonwoven fibrous web.

Eighth example embodiment: The embossed nonwoven fabric of any one of the first through seventh example embodiments, wherein: the mean thickness of the nonwoven fibrous web at the unembossed portion of the nonwoven fibrous web is no less than one hundred microns and no greater than about three hundred microns; and the mean thickness of the nonwoven fibrous web at the embossed portion of the nonwoven fibrous web is no less than five hundred microns and no greater than three thousand microns.

Nineth example embodiment: The embossed nonwoven fabric of any one of the first through eighth example embodiments, wherein the nonwoven fibrous web defines a plurality of apertures that extend through a thickness of the nonwoven fibrous web.

Tenth example embodiment: A product, comprising the embossed nonwoven fabric of any one of the first through nineth example embodiments, wherein the product is one of a diaper, a disposable undergarment, an incontinence pad, and a feminine hygiene pad.

Eleventh example embodiment: A nonwoven fabric, comprising a plurality of randomly oriented discrete spunbound polypropylene fibers and a plurality of randomly oriented discrete meltblown polypropylene fibers collectively forming a nonwoven fibrous web. A ratio of a mean diameter of the plurality of randomly oriented discrete spunbound polypropylene fibers to a mean diameter of the plurality of randomly oriented discrete meltblown polypropylene fibers is between 60:1 and 2:1. The plurality of randomly oriented discrete spunbound polypropylene fibers is present within the nonwoven fibrous web at no less than seventy percent by weight of the nonwoven fibrous web. The plurality of randomly oriented discrete meltblown polypropylene fibers is present within the nonwoven fibrous web at no less than two percent by weight of the nonwoven fibrous web and no greater than twenty percent by weight of the nonwoven fibrous web. A portion of the nonwoven fibrous web is embossed such that a mean thickness of the nonwoven fibrous web at an unembossed portion of the nonwoven fibrous web is no less than three times greater than a mean thickness of the nonwoven fibrous web at the embossed portion of the nonwoven fibrous web.

Twelfth example embodiment: The embossed nonwoven fabric of the eleventh example embodiment, wherein: the mean diameter of the plurality of randomly oriented discrete spunbound polypropylene fibers is no less than ten microns and no greater than twenty-two microns; and the mean diameter of the plurality of randomly oriented discrete meltblown polypropylene fibers is no less than a half micron and no greater than three microns.

Thirteenth example embodiment: The embossed nonwoven fabric of either the eleventh example embodiment or the twelfth example embodiment, wherein: the mean diameter of the plurality of randomly oriented discrete spunbound polypropylene fibers is no less than twelve microns and no greater than eighteen microns; and the mean diameter of the plurality of randomly oriented discrete meltblown polypropylene fibers is no less than seven-tenths of a micron and no greater than two microns.

Fourteenth example embodiment: The embossed nonwoven fabric of any one of the eleventh through thirteenth example embodiments, wherein the mean thickness of the nonwoven fibrous web at the unembossed portion of the nonwoven fibrous web is no less than five times greater than the mean thickness of the nonwoven fibrous web at the embossed portion of the nonwoven fibrous web.

Fifteenth example embodiment: The embossed nonwoven fabric of any one of the eleventh through fourteenth example embodiments, wherein: the mean thickness of the nonwoven fibrous web at the unembossed portion of the nonwoven fibrous web is no less than one hundred microns and no greater than about three hundred microns; and the mean thickness of the nonwoven fibrous web at the embossed portion of the nonwoven fibrous web is no less than five hundred microns and no greater than three thousand microns.

Sixteenth example embodiment: The embossed nonwoven fabric of any one of the eleventh through fifteenth example embodiments, wherein the nonwoven fibrous web defines a plurality of apertures that extend through a thickness of the nonwoven fibrous web.

Seventeenth example embodiment: A method for forming an embossed nonwoven fabric, comprising: bonding a mixture of a plurality of randomly oriented discrete spunbound polypropylene fibers and a plurality of randomly oriented discrete meltblown polypropylene fibers to form a nonwoven fibrous web, the plurality of randomly oriented discrete spunbound polypropylene fibers present within the mixture at no less than seventy percent by weight of the mixture, the plurality of randomly oriented discrete meltblown polypropylene fibers present within the mixture at no less than two percent by weight of the mixture and no greater than twenty percent by weight of the mixture; and embossing a portion of the nonwoven fibrous web such that a mean thickness of the nonwoven fibrous web at an unembossed portion of the nonwoven fibrous web is no less than three times greater than a mean thickness of the nonwoven fibrous web at the embossed portion of the nonwoven fibrous web.

Eighteenth example embodiment: The method of the seventeenth example embodiment, wherein a speed of the nonwoven fibrous web during the embossing is no less than two hundred meters per second and no greater than one thousand meters per second.

Nineteenth example embodiment: The method of either of the seventeenth example embodiment or the eighteenth example embodiment, wherein a ratio of a mean diameter of the plurality of randomly oriented discrete spunbound polypropylene fibers to a mean diameter of the plurality of randomly oriented discrete meltblown polypropylene fibers being between 50:1 and 3:1.

1. A nonwoven fabric, comprising:
a first plurality of randomly oriented discrete fibers and a second plurality of randomly oriented discrete fibers mixed and bonded together to collectively form a nonwoven fibrous web,
wherein a ratio of a mean diameter of the first plurality of randomly oriented discrete fibers to a mean diameter of the second plurality of randomly oriented discrete fibers is between 50:1 and 3:1,
wherein the first plurality of randomly oriented discrete fibers is present within the nonwoven fibrous web at no less than seventy percent by weight of the nonwoven fibrous web,
wherein the second plurality of randomly oriented discrete fibers is present within the nonwoven fibrous web at no less than two percent by weight of the nonwoven fibrous web and no greater than twenty percent by weight of the nonwoven fibrous web, and
wherein a portion of the nonwoven fibrous web is embossed.

2. The nonwoven fabric of claim 1, wherein the first plurality of randomly oriented discrete fibers are polypropylene fibers, and the second plurality of randomly oriented discrete fibers are polypropylene fibers.

3. The nonwoven fabric of claim 1, wherein the first plurality of randomly oriented discrete fibers are spunbound polypropylene fibers, and the second plurality of randomly oriented discrete fibers are meltblown polypropylene fibers.

4. The nonwoven fabric of claim 1, wherein:
the mean diameter of the first plurality of randomly oriented discrete fibers is no less than ten microns and no greater than twenty-two microns; and
the mean diameter of the second plurality of randomly oriented discrete fibers is no less than a half micron and no greater than three microns.

5. The nonwoven fabric of claim 1, wherein:
the mean diameter of the first plurality of randomly oriented discrete fibers is no less than twelve microns and no greater than eighteen microns; and
the mean diameter of the second plurality of randomly oriented discrete fibers is no less than seven-tenths of a micron and no greater than two microns.

6. The nonwoven fabric of claim 1, wherein a mean thickness of the nonwoven fibrous web at an unembossed portion of the nonwoven fibrous web is no less than three times greater than a mean thickness of the nonwoven fibrous web at the embossed portion of the nonwoven fibrous web.

7. The nonwoven fabric of claim 6, wherein the mean thickness of the nonwoven fibrous web at the unembossed portion of the nonwoven fibrous web is no less than five times greater than the mean thickness of the nonwoven fibrous web at the embossed portion of the nonwoven fibrous web.

8. The nonwoven fabric of claim 6, wherein:
the mean thickness of the nonwoven fibrous web at the unembossed portion of the nonwoven fibrous web is no less than one hundred microns and no greater than about three hundred microns; and
the mean thickness of the nonwoven fibrous web at the embossed portion of the nonwoven fibrous web is no less than five hundred microns and no greater than three thousand microns.

9. The nonwoven fabric of claim 1, wherein the nonwoven fibrous web defines a plurality of apertures that extend through a thickness of the nonwoven fibrous web.

10. A product, comprising the embossed nonwoven fabric of claim 1, wherein the product is one of a diaper, a disposable undergarment, an incontinence pad, and a feminine hygiene pad.

11. A nonwoven fabric, comprising:
a plurality of randomly oriented discrete spunbound polypropylene fibers and a plurality of randomly oriented discrete meltblown polypropylene fibers mixed and bonded together to collectively form a nonwoven fibrous web,
wherein a ratio of a mean diameter of the plurality of randomly oriented discrete spunbound polypropylene fibers to a mean diameter of the plurality of randomly oriented discrete meltblown polypropylene fibers is between 60:1 and 2:1,
wherein the plurality of randomly oriented discrete spunbound polypropylene fibers is present within the nonwoven fibrous web at no less than seventy percent by weight of the nonwoven fibrous web,
wherein the plurality of randomly oriented discrete meltblown polypropylene fibers is present within the nonwoven fibrous web at no less than two percent by weight of the nonwoven fibrous web and no greater than twenty percent by weight of the nonwoven fibrous web, and
wherein a portion of the nonwoven fibrous web is embossed such that a mean thickness of the nonwoven fibrous web at an unembossed portion of the nonwoven fibrous web is no less than three times greater than a mean thickness of the nonwoven fibrous web at the embossed portion of the nonwoven fibrous web.

12. The nonwoven fabric of claim 11, wherein:
the mean diameter of the plurality of randomly oriented discrete spunbound polypropylene fibers is no less than ten microns and no greater than twenty-two microns; and
the mean diameter of the plurality of randomly oriented discrete meltblown polypropylene fibers is no less than a half micron and no greater than three microns.

13. The nonwoven fabric of claim 11, wherein:
the mean diameter of the plurality of randomly oriented discrete spunbound polypropylene fibers is no less than twelve microns and no greater than eighteen microns; and
the mean diameter of the plurality of randomly oriented discrete meltblown polypropylene fibers is no less than seven-tenths of a micron and no greater than two microns.

14. The nonwoven fabric of claim 11, wherein the mean thickness of the nonwoven fibrous web at the unembossed portion of the nonwoven fibrous web is no less than five times greater than the mean thickness of the nonwoven fibrous web at the embossed portion of the nonwoven fibrous web.

15. The nonwoven fabric of claim 11, wherein:
the mean thickness of the nonwoven fibrous web at the unembossed portion of the nonwoven fibrous web is no less than one hundred microns and no greater than about three hundred microns; and
the mean thickness of the nonwoven fibrous web at the embossed portion of the nonwoven fibrous web is no less than five hundred microns and no greater than three thousand microns.

16. The nonwoven fabric of claim 11, wherein the nonwoven fibrous web defines a plurality of apertures that extend through a thickness of the nonwoven fibrous web.

17. A method for forming an embossed nonwoven fabric, comprising:
bonding a mixture of a plurality of randomly oriented discrete spunbound polypropylene fibers and a plurality of randomly oriented discrete meltblown polypropylene fibers to form a nonwoven fibrous web, the plurality of randomly oriented discrete spunbound polypropylene fibers present within the mixture at no less than seventy percent by weight of the mixture, the plurality of randomly oriented discrete meltblown polypropylene fibers present within the mixture at no less than two percent by weight of the mixture and no greater than twenty percent by weight of the mixture; and
embossing a portion of the nonwoven fibrous web such that a mean thickness of the nonwoven fibrous web at an unembossed portion of the nonwoven fibrous web is no less than three times greater than a mean thickness of the nonwoven fibrous web at the embossed portion of the nonwoven fibrous web.

18. The method of claim 17, wherein a speed of the nonwoven fibrous web during the embossing is no less than two hundred meters per second and no greater than one thousand meters per second.

19. The method of claim 17, wherein a ratio of a mean diameter of the plurality of randomly oriented discrete spunbound polypropylene fibers to a mean diameter of the plurality of randomly oriented discrete meltblown polypropylene fibers being between 50:1 and 3:1.
